(19) United States
(12) Patent Application Publication
(10) Pub. No.: US 2011/0218141 A1
(43) Pub. Date: Sep. 8, 2011

(54) Leptin therapy to increase muscle mass and to treat muscle wasting conditions

(76) Inventor: Mark W. Hamrick, Augusta, GA (US)

(21) Appl. No.: 12/932,704

(22) Filed: Mar. 3, 2011

Related U.S. Application Data
(60) Provisional application No. 61/339,361, filed on Mar. 3, 2010.

(51) Int. Cl. A61K 38/22; A61P 21/00

(52) U.S. Cl. 514/4.9

(57) ABSTRACT

The present invention provides a method of treating a subject suffering from a muscle wasting disorder comprising the step of administering to the subject an effective dose of leptin, leptin analog or leptin derivative.

### CROSS-REFERENCE TO RELATED APPLICATION

This nonprovisional application claims benefit of priority under 35 U.S.C. §119(e) of provisional applications U.S. Ser. No. 61/339,361, filed Mar. 3, 2010, now abandoned, the entirety of which is hereby incorporated by reference.

### FEDERAL FUNDING LEGEND

This invention was made with government support under NIAMS AR 049717 awarded by National Institutes of Health and under PR093619 awaded by the US Army Medical Research and Material Command. The government has certain rights in the invention.

### BACKGROUND OF THE INVENTION

1. Field of the Invention

The present invention relates to the field of muscle physiology. More specifically, the present invention relates to, inter alia, methods for using leptin to increase muscle mass and to, inter alia, treat patients with muscle wasting disorders.

2. Description of the Related Art

It is estimated that approximately 5-8% of muscle mass is lost per decade of life after about age 30, and this rate of decline accelerates after age 65 (Greenlund and Nair, 2003). Age-related muscle atrophy, or sarcopenia, significantly increases mortality among the elderly because of muscle weakness and postural instability, which are the major causes of falls and fractures (Nguyen et al., 2005; Jarvinen et al., 2008).

The cytokine-like hormone leptin is an important factor linking food intake with energy expenditure and body composition (Flier, 1998; Hamrick, 2004). Leptin is secreted from fat cells (adipocytes), but muscle is also a primary source of leptin (Wang et al., 1998), and serum leptin levels increase with increased muscle mass (Fernandez-Real et al., 2000). Older populations of frail, continuing care patients, e.g., individuals over the age of 85, were observed to show low serum leptin, low bone mass, and muscle atrophy (Hubbard et al., 2007). Leptin deficiency itself is associated with decreased muscle mass (Hamrick et al., 2004), and the functional characteristics of skeletal muscle in leptin-deficient ob/ob mice resemble those of aged rodents (Warmington et al., 2000). Leptin receptors are abundantly expressed in peripheral tissues such as skeletal muscle, liver, and bone (Margetic et al., 2002). Leptin receptors have been identified in human skeletal muscle (Guerra et al., 2007), their expression is elevated with disuse atrophy (Chen et al., 2007), and leptin-deficiency increases expression of the muscle-wasting protein myostatin (Allen et al., 2008).

Recent studies suggest that alterations in the expression of muscle-specific microRNAs (miRNAs) may play a role in several muscle disorders. MicroRNAs are short (˜22 nucleotides) molecules that bind to complementary sequences of specific target mRNAs and inhibit translation. Accumulating evidence indicates that miRNAs regulate essential biological functions, such as cellular differentiation, proliferation, and apoptosis, and have become one of the most important gene regulators in eukaryotic organisms (e.g., Hatfield et al., 2005; Plasterk, 2006; He et al., 2009).

A number of miRNAs have been identified that are tissue-specific, and may therefore be involved in tissue development, disease, and regeneration. For example, the expression of the muscle-specific miRNA miR-206 has been found to decrease with aging and increase with mechanical stimulation (Drummond et al., 2008). Local injection of miR-206 can accelerate muscle regeneration (Nakasa et al., 2009), whereas miR-133 promotes myoblast proliferation and miR-1 can suppress myoblast proliferation (van Rooij et al., 2008). A broad molecular profiling approach examining the expression patterns of miRNAs in primary muscular disorders identified 18 miRNAs that were associated with specific diseases such as Duchenne and Becker muscular dystrophies (Eisenberg et al., 2007). These findings suggest that miRNAs may represent potential therapeutic targets for muscle-related diseases (Chen, et al., 2009).

Muscle wasting refers to the progressive loss of muscle mass and/or to the progressive weakening and degeneration of muscles, including skeletal or voluntary muscles, cardiac muscles controlling the heart (cardiomyopathias), and smooth muscles. Chronic muscle wasting is a condition (i.e., persisting over a long period of time) characterized by progressive loss of muscle mass, as well as muscle weakening and degeneration. The loss of muscle mass occurs during catabolic muscle protein degradation.

Muscle wasting is associated with developing of chronic, neurological, genetic or infectious pathologies. These conditions are: cardiomyopathy, Duchenne and myotonic skeletal muscle dystrophies, muscle atrophies including partial post-polio muscle atrophy, cachexias such as cardiac, AIDS- or cancer-caused, malnutrition, leprosy, diabetes, renal disease, chronic obstructive pulmonary disease, cancer, late stage renal failure, sarcopenia, emphysema, and osteomalacia.

Some other conditions may cause muscle wasting. Such conditions include alcoholism, chronic lower back pain, advanced age, damage to central nervous system, peripheral nerve injury, chemical injury, extended burns, disuse atrophy and long term hospitalization with extremity(ies) immobilized.

Muscle wasting, if left without treatment, can be dangerous for overall well being. For example, the changes that occur during muscle wasting can be detrimental to an individual's health increasing susceptibility to infraction and poor performance status. Therefore innovative approaches are essential for basic science and in clinical applications to treat muscle wasting.

Thus, there is a lack in the prior art of improved methods and therapies to treat skeletal muscle disorders and diseases accompanied with muscle wasting. The present invention fulfills this long-standing need and desire in the art.

### SUMMARY OF THE INVENTION

The present invention teaches that loss of skeletal muscle mass with age is associated with marked changes in the expression of muscle-specific miRNAs. The present invention also determined that recombinant leptin therapy can increase muscle mass and myofiber hypertrophy in an animal model, and that leptin treatment can alter the miRNA expression profile that accompanies age associated muscle atrophy.

Thus, the present invention is directed to a method of treating a subject suffering from a muscle wasting disorder comprising the step of administering to said subject an effective dose of leptin, leptin derivative, or leptin analog.

In another embodiment, the present invention provides a method of stimulating muscle growth in a subject in need of such stimulation, comprising the step of administering to a subject an effective amount of a leptin analog agonist, or a pharmaceutically acceptable salt thereof, wherein said effective amount is at least an amount sufficient to produce a detectable increase in muscle growth.

Other and further aspects, features and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIGS. 1A-1E show the effect of leptin on muscle. Data is shown of the effect on body weight (FIG. 1A), quadriceps mass (FIG. 1B), quadriceps mass normalized by body weight (FIG. 1C), and cross-sectional area of extensor digitorum longus muscle fibers (FIG. 1D) in adult (12 mo) and aged mice (24 months) receiving saline (con) or recombinant leptin (lep; 10 ug/day). FIG. 1E shows cryostat sections of the extensor digitorum longus (EDL) stained with a Cy3-conjugated anti-laminin antibody from adult mice receiving saline (12 months), aged mice receiving saline (24 months+veh), and aged mice treated with leptin (24 months+lep). Note the relatively larger size of the extensor digitorum longus fibers in aged mice receiving leptin compared to the fibers of aged mice receiving saline.

FIG. 2A-2B show changes in miRNA expression. FIG. 2A show heat map and FIG. 2B shows miRNA expression changes >1-fold in quadriceps muscles from aged mice (24 months) compared to adult mice (12 months).

FIGS. 3A-3B show miRNA miRNA expression after leptin treatment. More specifically, FIG. 3A show Heat map and FIG. 3B shows miRNA expression changes >1-fold in quadriceps muscles from leptin-treated aged mice compared to vehicle (saline)-treated aged mice.

### DETAILED DESCRIPTION OF THE INVENTION

The present invention is directed to a method of treating a subject suffering from a muscle wasting disorder comprising the step of administering to said subject an effective dose of leptin, leptin analog or leptin derivative. Generally, there is a need in the art for an effective treatment of muscle wasting disorders developed due to, for example, a pathology, an illness, a disease or a condition which could be neurological, infectious, chronic or genetic in origin. Representative examples of the pathology, illness, disease or condition include but are not limited to muscular dystrophy, a muscular atrophy, X-linked spinal-bulbar muscular atrophy or a cachexia, age-associated muscle wasting disorder; or a disuse deconditioning-associated muscle wasting disorder.

Generally, the leptin, leptin analog or leptin derivative may be adminstered in any acceptable form as is well known in the art including administering a pharmaceutical composition comprising said leptin, leptin analog or leptin derivative pharmaceutically acceptable salt, pharmaceutical product, hydrate, nitrogen-oxide, or any combination thereof; and a pharmaceutically acceptable carrier. In addition, the leptin, leptin analog or leptin derivative may be administered by any route desired, including but not limited to intravenously, intraarterially, or intramuscularly injecting to said subject said pharmaceutical composition in liquid form; subcutaneously implanting in said subject a pellet containing said pharmaceutical composition; or orally administering to said subject said pharmaceutical composition in a liquid or solid form.

The pharmaceutical composition may be in the form of a pellet, a tablet, a capsule, a solution, a suspension, an emulsion, an elixir, a gel, a cream, a suppository or a parenteral formulation. The amount of the leptin, leptin analog or leptin derivative administered would of course vary according to the size of the subject and various other factor but would typically be administered in a dose from about 0.01 mg/kg to about 100 mg/kg of the subject's body weight. It is contemplated that a subject who would benefit primarily from such treatment would have a leptin level of 1-5 ng/ml or less before administration of leptin, leptin analog, or leptin derivative. In one preferred embodiment, the leptin, leptin analog, or leptin derivative may be administered subcutaneously and is recombinant human leptin.

The present invention is further directed to a method for stimulating muscle growth in a subject in need of such stimulation, comprising the step of administering to a subject an effective amount of a leptin analog agonist, or a pharmaceutically acceptable salt thereof, wherein said effective amount is at least an amount sufficient to produce a detectable increase in muscle growth. For example, the subject may have a disease or disorder or undergoing a treatment accompanied by weight loss due to cachexia. Such cachexia may be related to, for example, anorexia, bulimia, cancer, AIDS or a chronic obstructive pulmonary disease. Representative treatments accompanied by weight loss include but are not limited to, chemotherapy, radiation therapy, temporary immobilization, permanent immobilization and dialysis.

As used herein, the term “a” or “an”, when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, method steps, and/or methods of the invention. It is contemplated that any device or method described herein can be implemented with respect to any other device or method described herein.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, the term “contacting” refers to any suitable method of bringing a compound or a composition into contact with a cell. In vitro or ex vivo, this is achieved by exposing the cell to the compound or agent in a suitable medium. For in vivo applications, any known method of administration is suitable as described herein.

As used herein, the term “subject” refers to any human or non-human recipient of the composition described herein.

A “therapeutically acceptable amount” or “effective amount” of a leptin or analog or derivative thereof, regardless of the formulation or route of administration, is that amount which elicits a desired biological response in a subject. The biological effect of the therapeutic amount may occur at and be measured at many levels in an organism. For example, the biological effect of the therapeutic amount may occur at and be measured at the cellular level by measuring the response at a receptor, which binds leptin and/or a leptin analog, or the biological effect of the therapeutic amount may occur at and be measured at the system level. The biological effect of the therapeutic amount may occur at and be measured at the organism level, such as the alleviation of a symptom(s) or progression of a disease or condition in a subject. A therapeutically acceptable amount of a compound or composition of the invention, regardless of the formulation or route of administration, may result in one or more biological responses in a subject. In the event that the compound or composition of the invention is subject to testing in an in vitro system, a therapeutically acceptable amount of the compound or composition may be viewed as that amount which gives a measurable response in the in vitro system of choice.

The present invention demonstrates that leptin treatment significantly increased hindlimb muscle mass and extensor digitorum longus fiber size in aged mice. These findings suggest that aging in skeletal muscles is associated with marked changes and that nutrient-related hormones such as leptin may be able to reverse muscle atrophy in aging skeletal muscle.

The aged C57BU6 mouse animal model shares a number of key features in common with the aging human musculoskeletal system: an age-related decline in serum leptin, decreased muscle mass, and loss of bone density (Hamrick et al., 2006). Using this aged C57BU6 mouse model, it was determined by the present invention that loss of skeletal muscle mass as related to the age in this model is associated with marked changes in the expression of muscle-specific miRNAs. Recombinant leptin also increased muscle mass and myofiber hypertrophy in this animal model, and leptin treatment altered the miRNA expression profile that accompanies age-associated muscle atrophy.

The following example(s) are given for the purpose of illustrating various embodiments of the invention and are not meant to limit the present invention in any fashion.

### Example 1

### Methodology for Leptin Treatment and Body/Muscle Weight Change Detection

Twelve and 24 months old C57BU6 mice were obtained from the National Institute on Aging (Taconic Farms). Twenty four mice of each age group were divided into the control group and those given subcutaneous injections of leptin (10 μg/day) for 10 days. Mice were euthanized after the 10^{th} day treatment period and body weights and quadriceps masses were recorded. The extensor digitorum longus muscles (EDL; predominantly type II, or fast-twitch fibers) and soleus muscles (primarily type I, or slow-twitching fibers) were released by dissection of the covering connective tissues, embedded in the OCT medium, and snap frozen. The frozen cryostat sections of the above muscles were stained with hematoxylene&eosine and muscle fiber cross-sectional areas were measured using SigmaScan.

### Example 2

### Effect of Leptin Administration on the Body/Muscle Weight and Muscle Morphology

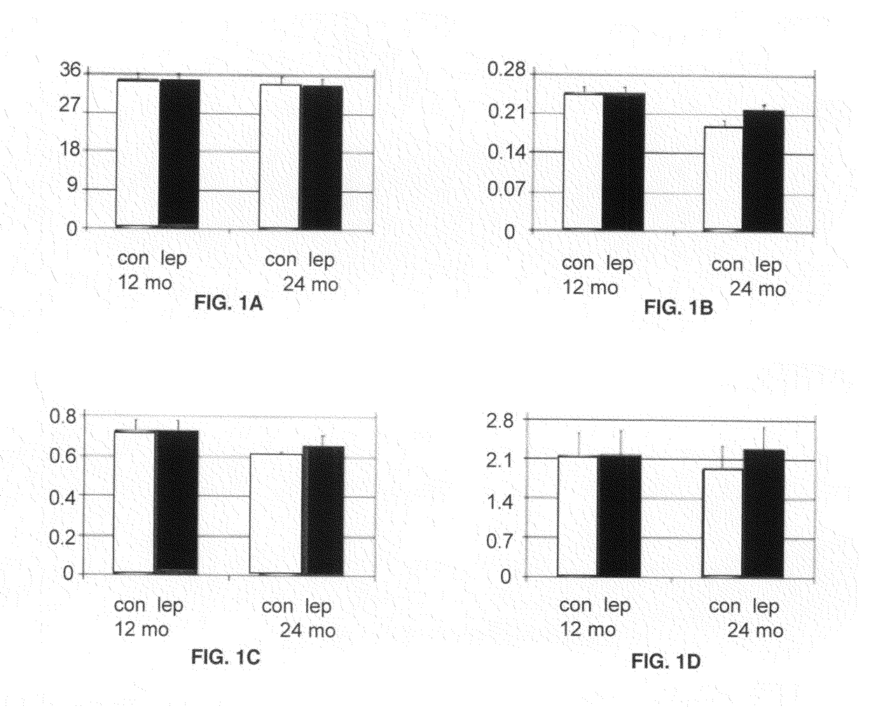

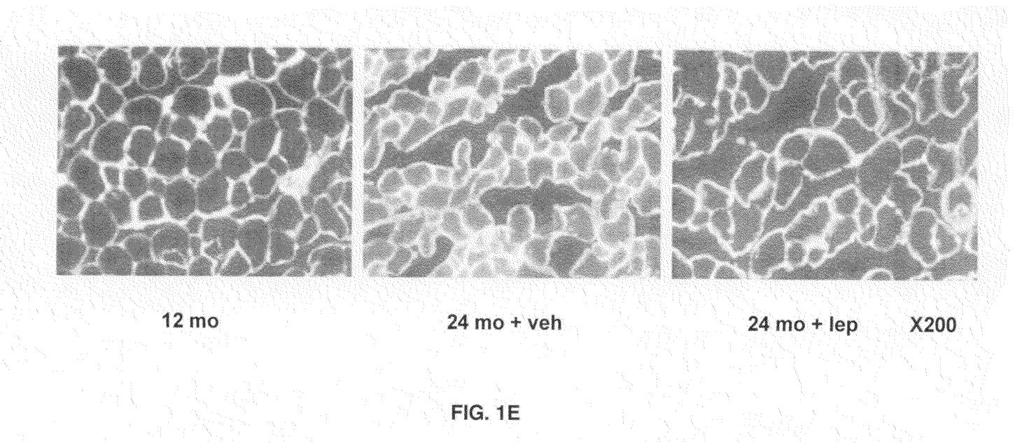

Body weight data demonstrate that the aged mice were significantly smaller than the younger mice, and that leptin treatment did not significantly alter body weight in mice of either age group (FIG. 1A). Quadriceps muscle weights were significantly lower in the aged mice, and were also slightly (but not significantly) lower when normalized to body weight (FIGS. 1B-1C).

Leptin treatment did, however, significantly increase quadriceps muscle mass both absolutely (FIG. 1B) and relative to body mass (FIG. 1C) in the aged mice but not in the younger mice. Muscle fiber cross-sectional areas of the extensor digitorum longus muscle (EDL) were slightly lower in aged mice, and leptin treatment significantly increased extensor digitorum longus fiber area in the aged mice but not the young mice (FIGS. 1D and E). Muscle fiber cross-sectional areas of the soleus muscle were similar between young and aged mice, and leptin treatment produced a slight but not significant increase in soleus fiber area in mice from each age group.

### Example 3

miRNAs in Aging of Muscle Development and Effect of Leptin Treatment on miRNAs Expression Patterns

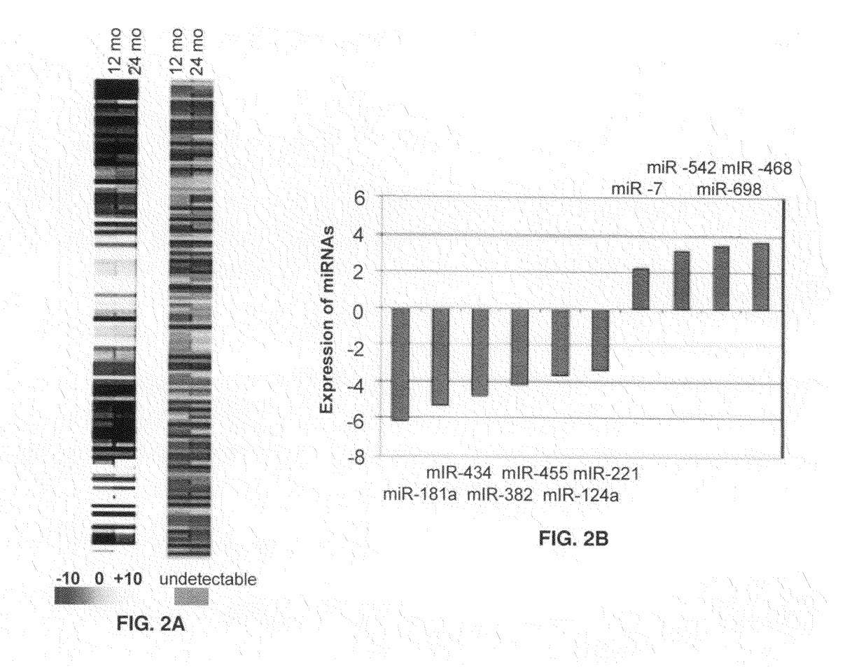

To test if miRNAs are involved in aging muscle development, the TaqMan RT-PCR miRNA array was first used to compare the miRNA expression profiles between mice 12- and 24-months of age. miRNA profilings revealed that relative expression of 57 miRNAs was significantly altered in quadriceps muscle samples from aged mice compared to those from younger adult mice. Approximately 36 miRNAs were decreased whereas only 21 miRNAs were increased (FIGS. 2A and 2B). The muscle-specific miRNA miR-206 was upregulated about 2-fold in aged mice compared to young.

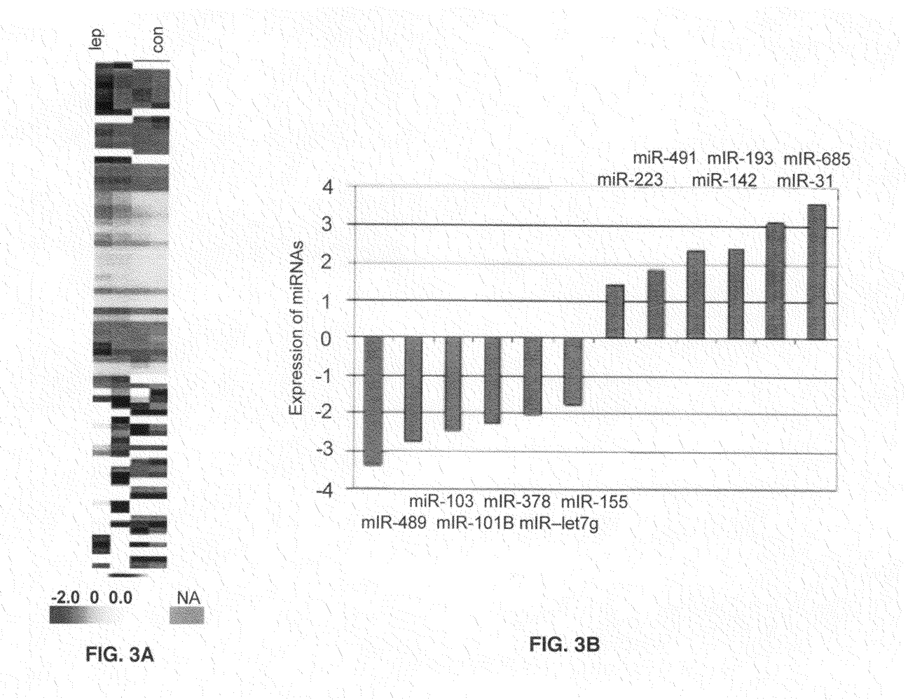

To test if the miRNA expression profile of aged skeletal muscle might be altered with leptin treatment, miRNA gene expression profiles in the muscles from vehicle- or leptin-treated mice were further analyzed. As shown in FIG. 3A, 37 genes were changed in leptin-treated aged mice compared to control aged mice, including 7 upregulated and 30 downregulated miRNAs. Interestingly, leptin treatment reversed the expression of several miRNAs in muscles from aged mice. miR685 and miR-142-3p were all downregulated in quadriceps muscles of aged mice compared to younger 12 month-old mice, whereas leptin treatment significantly increased the expression of these miRNAs relative to control aged mice (FIG. 3B). Similarly, leptin treatment also decreased expression of several miRNAs whose expression was increased in muscles of aged mice compared to muscles from younger mice. miR-155 was increased in muscle of aged mice, but leptin treatment significantly decreased miR-155 expression compared to vehicle-treated controls (FIG. 3B).

Discussion of miRNA Regulation Changes in Skeletal Muscles of Leptin-Treated Animals

To date, the tissue-specific expression of three miRNAs—miR-1, miR-133a, and miR-206—has been consistently associated with skeletal muscle. These miRNAs have also been shown to induce significant effects on muscle development and myogenesis by targeting myogenic factors such as mef2, SRF, and myostatin (Chen, et al., 2009). Interestingly, this profiling approach reveals that miRNA miR-206 was significantly up-regulated with age in the mice (FIG. 2). miR-206 can induce muscle hypertrophy (Nakasa, et al., 2009), and its increased expression with muscle atrophy in aging may indicate an adaptive, compensatory response to antagonize other catabolic signals. The data presented here also identify additional miRNAs that may be involved in age-associated muscle atrophy. Specifically, miR-698 and -468 were highly upregulated with age and miR-434, -455, treatment include miRNAs previously identified as playing a role in muscle regeneration. Greco et al. (2009) demonstrated that specific miRNAs were upregulated in the inflammatory (miR-222, -223), degenerative (miR-1, -29c, -135a), and regenerative (miR-206, -34c, -31, -335, -449, and -494) phases of muscle damage and regeneration in Duchenne muscular dystrophy. miR-223 and miR-31 were both upregulated in skeletal muscles from leptin-treated aged mice (FIG. 3), suggesting that leptin can activate molecular pathways involved in muscle repair and regeneration. miRNAs that were downregulated in skeletal muscles from leptin-treated animals include several that have been previously described as playing role in mesenchymal stem cell differentiation. These include miR-489, known to be downregulated during the osteogenic differentiation of mesenchymal stem cell (Schoolmeesters, et al., 2009), and miR-103, which is specifically localized to bone marrow populations of mesenchymal stem cells (Liu, et al., 2009) and can induce adipogenic differentiation when expressed ectopically (Xie, et al., 2009). The strong downregulation of these genes associated with mesenchymal stem cell suggests that exogenous leptin may significantly alter the expression profile of muscle-derived stem cells.

Thus, the present invention reveals that as C57BL6 mice age they lose skeletal muscle mass, and are therefore a useful animal model for studying the development of age-associated pathologies of the musculoskeletal system such as osteoporosis and sarcopenia (Hamrick et al., 2006). Loss of muscle mass with age in these mice resembles human age-associated muscle loss in that fast-twitch fibers, which are abundant in the extensor digitorum longus, are reduced in size more so than slow-twitch fibers, which are more numerous in the soleus muscle. Age-associated loss of muscle mass in this mouse model was accompanied by specific changes in the expression pattern of miRNAs. miRNA expression data herein indicates that several miRNAs are altered with aging and may contribute to a decreased proliferative potential of myogenic precursors and a tendency toward terminal myogenic differentiation. While leptin therapy increased muscle mass in the aged mice, it can regulate 37 miRNA gene expression, but only reverse three dysregulated miRNAs in aged mice, miR-685, miR-142-3p, and miR-155, suggesting that other therapeutic approaches need to be investigated in order to target certain miRNAs identified in aged muscle.

The present invention provides evidence showing that a nutrient-related peptide (leptin) has anabolic effects in aging skeletal muscle. Leptin treatment increased the relative mass of quadriceps muscles in aged mice as well as the fiber size of skeletal muscle fibers in the extensor digitorum longus (FIG. 1). These results are consistent with previous work in leptin-deficient ob/ob mice, where recombinant leptin therapy suppresses myostatin and Foxo3a expression (Allen et al., 2008; Sainz et al., 2009). As noted, populations of aging adults show low serum leptin levels (Hubbard et al., 2007), and aging in mice is accompanied by a decline in serum leptin (Hamrick et al., 2006). Previous work (e.g., Fernandez-Galaz et al., 2002) has indicated that leptin resistance, via downregulation of central (hypothalamic) leptin receptors, increases with age.

The present invention suggests that, at least in the case of skeletal muscle, leptin resistance may not necessarily increase with age. That is, exogenous leptin is capable of inducing a significant anabolic response in skeletal muscle, as well as producing changes in the expression of specific miRNAs. MicroRNAs increased in aged muscle with leptin treatment include miRNAs previously identified as playing a role in muscle regeneration. Greco et al (2009) demonstrated that specific miRNAs were upregulated in the inflammatory (miR-miR-222, -223), degenerative (miR-1, -29c, -135a), and regenerative (miR206, -34c, -31, -335, 449, and -494) phases of muscle damage and regeneration in Duchenne muscular dystrophy. miR-223 and miR-31 were both upregulated in skeletal muscles from leptin-treated aged mice (data not shown), suggesting that leptin can activate molecular pathways involved in both muscle repair and regeneration.

miRNAs that were downregulated in skeletal muscles isolated from leptin-treated animals included several differences that have been previously described as playing a role in mesenchymal stem cell differentiation. Those included miR-489, known to be downregulated during the osteogenic differentiation of mesenchymal stem cells (Schoolmeesters et al., 2009), and miR-103, which is specifically localized to bone marrow populations of mesenchymal stem cells (Liu et al., 2009) and can induce adipogenic differentiation when expressed ectopically (Xie et al., 2009). The strong downregulation of these genes associated with mesenchymal stem cells suggests that exogenous leptin may significantly alter the expression profile of muscle-derived stem cells.

The following references may have been cited herein:
Allen, et al., 2008. Am J Phys Endocrinol Metab 294, E918-27.
Cardinali, et al., 2009, PLoS One 4, e7607.
Chapman, I M., 2004, Best Pract Res Clin Endocrinol Metab. 18, 437-52.
Chen, et al., 2007, Physiol Genomics 31, 510-520.
Chen, et al., 2009, J Cell Science 122, 13-20.
Drummond, et al., 2008, Am J Physiol Endrocrinol Metab 295: E1333-40.
Eisenberg, et al., 2007, Proc Natl Acad Sci (USA) 104, 17016-17021.
Fernandez-Real, et al., 2000. Eur J Endocrinol 142, 25-29.
Fernández-Galaz, et al., 2002, Diabetologia 45, 997-1003.
Flier, J., 1998. J Clin Endocrinol Metabol 83, 1407-1413.
Giresi, et al., 2005. Physiol Genomics 21, 253-63.
Greco, S., DeSimone, M., Colussi, C., Zaccagnini, G., Fasanaro, P., Pescatori, M., Cardani, et al., 2009 FASEB J 23, 3335-46.
Greenlund, 2003. Mech Ageing Dev 124, 287-99
Guerra, et al., 2007, J Appl Physiol 102, 17861792.
Hamrick, M. W., 2004. J Bone Miner Res 19, 1607-1611
Hamrick, et al., 2004 Bone 34, 376-383.

Hamrick, et al., 2006. Bone 39, 845-853.
Hatfield, et al., 2005. Nature. 435, 974-978.
He, et al., 2009 J Cell Mol Med 13, 606-18.
Hubbard, et al., 2008. JAGS 56, 279-284.
Jarvinen, et al., 2008. BMJ 336, 124-126.
Liu, et al., 2009. Cell Transplant 18: 1039-45.
Margetic, et al., 2002. Int J Obes Rel Metab Disord 26, 1407-33.
Miyake, 2009. J Biol Chem 294, 19679-93.
Nakasa, et al., 2009. J Cell Mol Med, September 14 [Epub ahead of print].
Nguyen, et al., 2005. J Bone Miner Res 20, 1921-1928.
Nomura, et al., 2008. Biochem Biophys Res Comm 365, 863-869.
Plasterk, R., 2006. MicroRNAs in animal development. Cell 124, 877-881.
Sainz, et al., 2009 PLoS One 4, e6808.
Schoolmeesters, et al., 2009. PLoS One 4, e5606.
Stolzing, A., cuff, A., 2006. Aging Cell 5, 213-224.
Tsuchida, et al., 2008 Endocr J 55, 11-21.
Van Rooij, et al., 2008. Trends Genetics 24, 159-66.
Wallace, J., 1999. Malnutrition and enteral/parenteral alimentation, In: Hazzard et al., editors. Principles of geriatric medicine and gerontology, 4th ed. New York: McGraw-Hill, 1455-69.
Wang, et al., 1998. Nature 393, 684-688.
Warmington, et al., 2000. Int J Obes Rel Metab Disord 24, 1040-1050.
Xie, et al., 2009. Diabetes 58, 1050-57.
Zhang, et al., 2008. J Bone Miner Res 23, 1118-28.

Any patents or publications mentioned in this specification are indicative of the levels of those skilled in the art to which the invention pertains. These patents and publications are incorporated by reference herein to the same extent as if each individual publication was incorporated by reference specifically and individually. One skilled in the art will appreciate that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

What is claimed is:

1. A method of treating a subject suffering from a muscle wasting disorder comprising the step of administering to said subject a effective dose of leptin, leptin analog or leptin derivative.

2. The method of claim 1, wherein said muscle wasting disorder is due to a pathology, an illness, a disease or a condition.

3. The method of claim 2, wherein said pathology, illness, disease or condition is neurological, infectious, chronic or genetic.

4. The method of claim 2, wherein said pathology, illness, disease or condition is a muscular dystrophy, a muscular atrophy, X-linked spinal-bulbar muscular atrophy or a cachexia.

5. The method according to claim 1, wherein said muscle wasting disorder is an age-associated muscle wasting disorder; or a disuse deconditioning-associated muscle wasting disorder.

6. The method according to claim 1, wherein said muscle wasting disorder is a chronic muscle wasting disorder.

7. The method according to claim 1, wherein said administering comprises administering a pharmaceutical composition comprising said leptin, leptin analog or leptin derivative, pharmaceutically acceptable salt, pharmaceutical product, hydrate, nitrogen-oxide, or any combination thereof; and a pharmaceutically acceptable carrier.

8. The method according to claim 1, wherein said administering comprises intravenously, intraarterially, or intramuscularly injecting to said subject said pharmaceutical composition in liquid form; subcutaneously implanting in said subject a pellet containing said pharmaceutical composition; or orally administering to said subject said pharmaceutical composition in a liquid or solid form.

9. The method according to claim 8, wherein said pharmaceutical composition is a pellet, a tablet, a capsule, a solution, a suspension, an emulsion, an elixir, a gel, a cream, a suppository or a parenteral formulation.

10. The method of claim 1, wherein said composition is administered in a dose of from about 0.01 mg/kg to about 100 mg/kg of the subject's body weight.

11. The method of claim 1, wherein said composition is administered by a route selected from the group consisting of systemic, oral, intravenous, intramuscular, subcutaneous, intraorbital, intranasal, intracapsular, intraperitoneal, intracisternal, intratracheal, intraarticular administration, and by absorption through the skin.

12. The method of claim 1, wherein said leptin, leptin analog or leptin derivative is administered together with a pharmaceutically acceptable carrier.

13. The method of claim 1, wherein the subject has a leptin level of 1 ng/ml or less before administration of leptin, leptin analog, or leptin derivative.

14. The method of claim 1, wherein the subject has a leptin level of 5 ng/ml or less before administration of leptin, leptin analog, or leptin derivative.

15. The method of claim 1, wherein said leptin, leptin analog, or leptin derivative is administered subcutaneously.

16. The method of claim 1, wherein said leptin is recombinant human leptin.

17. A method for stimulating muscle growth in a subject in need of such stimulation, comprising the step of administering to a subject an effective amount of a leptin analog agonist, or a pharmaceutically acceptable salt thereof, wherein said effective amount is at least an amount sufficient to produce a detectable increase in muscle growth.

18. The method of claim 17, wherein subject has a disease or disorder or undergoing a treatment accompanied by weight loss.

19. The method according to claim 18, wherein said weight loss is due to onset of cachexia.

20. The method according to claim 18, wherein said cachexia is incidental to said subject suffering from anorexia, bulimia, cancer, AIDS or chronic obstructive pulmonary disease.

21. The method according to claim 18, wherein said weight loss is due to the onset of a wasting syndrome.

22. The method according to claim 18, wherein said treatment accompanied by weight loss is selected from the group consisting of chemotherapy, radiation therapy, temporary immobilization, permanent immobilization and dialysis.

23. The method of according to claim 17, wherein said subject in need thereof is not suffering from a disease or disorder and is not undergoing a treatment accompanied by weight loss and is otherwise healthy.
